(19) United States
(12) Patent Application Publication
(10) Pub. No.: US 2018/0206733 A1
(43) Pub. Date: Jul. 26, 2018

(54) DEVICE, METHOD AND SYSTEM FOR MONITORING AND MANAGEMENT OF CHANGES IN HEMODYNAMIC PARAMETERS

(71) Applicants: Michael Kasan, Atlit (IL); Or Goshen, Haifa (IL)

(72) Inventors: Michael Kasan, Atlit (IL); Or Goshen, Haifa (IL)

(21) Appl. No.: 15/412,159

(22) Filed: Jan. 23, 2017

(51) Int. Cl. A61B 5/024; A61B 5/021; A61B 5/1455; A61B 5/00

(52) U.S. Cl. CPC A61B 5/024; A61B 5/021; A61B 5/6813; A61B 5/0002; A61B 5/4875; A61B 5/14551

(57) ABSTRACT

A method, device and system monitoring and management of a person's health by measuring changes in hemodynamic parameters is provided. The method includes the steps of inputting personal data into a device worn by a user; calibrating the personal data to set a base line for analysis, monitoring changes in health-related data of the user, recorded by the device and transmitting the health-related data for analysis' analyzing the health-related data for changes in hemodynamic parameters and outputting the values based on the analysis, thereby allowing a diagnosis of the user's health to be made from the output values.

### FIELD OF THE INVENTION

The present invention relates to the monitoring and management of heart changes of hemodynamic parameters in general and to the monitoring of CO and blood pressure to manage a person's health and reduce hydration and Cardiac Output (CO) and other parameters by an algorithm that analyzes the PPG wave generated by generic off-the-shelf wearable devices and pulse oximeters.

### BACKGROUND OF THE INVENTION

Most (if not all) wearable devices measure the same parameters: steps, distance and some of them can measure heart rate. The fact that only these parameters are measured, confines the solution to the fitness market.

The market and manufacturers are looking for technology that will provide their devices with added value. Being able to measure additional, more medical parameters, will provide the players with added value—the ability to stand out against competition and be able to market the solution to additional markets such as the wellness and medical.

The ability to monitor hemodynamic parameters with an easy-to-use, available and inexpensive wearable device (bracelet, ring oximeter, handheld devices or pulse oximeter) is a game changer. Any technology, that can utilize the same wearable devices without the need to manufacture expensive devices will make this solution accessible to large markets.

For example:
Patients with heart issues like congestive heart failure that need monitoring on a daily basis from anywhere (clinics home while traveling). Better monitoring can decrease readmission and hospitalization.
Patients who need ongoing monitoring (hypertension). Better monitoring will result in better disease management
Employees who are undergoing a wellness program and need additional parameters to be monitored—thus maintain a healthier life style
Armed forces and law enforcement personal who need their parameters constantly monitored
Fitness patients who want better monitoring of their parameters

There are many more examples and markets. Each market reflects a big business opportunity and the ability to improve the quality of life of numerous numbers of customers and patients.

Today, there is no easy and available method to monitor hemodynamic parameters—especially once you leave a medical facility

With a prevalence of 5.8 million in the US alone (2012), heart failure (HF) is a common syndrome associated with substantial morbidity, mortality, and health-care expenditures. Close to 1 million HF hospitalizations occur annually in the United States, with the majority of these resulting from worsening congestion in patients previously diagnosed with HF. An estimated 37.2 billion dollars is spent each year on HF in the United States. HF is the leading cause of hospitalization in individuals 65 years of age and older. One third of individuals who are hospitalized for HF either die or require readmission within 60 days. Earlier identification and treatment of congestion together with improved care coordination, management of co-morbid conditions, and enhanced patient self-management may help to prevent hospitalizations in patients with chronic HF. Such home monitoring extends from the promotion of self-care and home visitations, to telemedicine and remote monitoring of external or implantable devices.

The incidence of HF increases with age. According to the Centers for Disease Control, among the U.S. residents who have HF, 70 percent are 60 years of age or older. It is estimated that in 2020, 16.5 percent will be in this age group which will lead to a significant increase in the prevalence of HF is expected in coming years. According to the Centers for Disease Control, more than 20 percent of men will develop HF within six years of having a heart attack. An even higher percentage (more than 40 percent) of women will suffer from HF within that period of time after having a heart attack. Together, the aging of the U.S. population and an improved medical outlook for heart attack victims account for the approximate threefold increase in the yearly incidence of HF that has been observed over the past 10 years.

These statistics emphasize the need to develop and implement more effective strategies to assess, monitor, and treat heart failure. Interventions geared towards identifying and monitoring sub-clinical congestion would be of value in the home management of chronic HF. Recent studies show that hospitals fail in decreasing the “30 days readmission” rate, and recently more than 2000 hospitals were penalized for that. Although Medicare is willing to employ companies for disease management of HF and DM, 5 out 8 companies that treated 240,000 patients, decided to discontinue their participation as they weren't successful in reducing costs, compared to the baseline before the program started.

It is assumed that a major factor in this failure of the hospitals and the disease management companies was the lack of suitable tools to continuously monitor the health state of the patients in real time and to administer suitable interventions, especially low-cost interventions of Lifestyle to complement medication and keep the patients at their homes with reasonable quality of life.

The gold standard for Cardiac-Output is an invasive procedure called Thermodilution (Swan-Ganz). There is also a non-invasive measurement using echocardiography and PC-MRI, but they are expensive, require skilled doctor and confined to the hospital. So is Edwards Lifescience Vigileo Flow-Trac that is based on invasive BP (A-Line).

The non-invasive devices based on Impedance Cardio-Graph (ICG) are estimating the changes in fluid in the chest using 6 to 8 electrodes. Systems like that are offered by Cheetah NICOM and PhysioFlow (see pictures below). Another device is offered by BMEye (that was recently acquired by Edwards LifeScience).

The main problem of the existing invasive and non-invasive devices mentioned above is that none of them is suitable for using by the patient at home and that their cost is prohibitive.

The cost is many thousands and even tens of thousands dollars, they require skilled healthcare giver to place them on the patient and all of them need disposable sensors that are unacceptable for daily use at home. In addition, all of them interfere with the daily life of the patient.

Another major problem that makes them not practical for large-scale disease management is that all of them provide raw data that needs a skilled doctor interpretation. Just dumping continuous streams of data on the doctor is a useless strategy, as it does not have real economic advantage.

### SUMMARY OF THE INVENTION

The present invention is directed to a mobile wearable device that monitors changes in Blood Pressure, hydration, Cardiac Output (CO)/Cardiac Index (CI) and other hemodynamic parameters. The invention relates to generic (off-the-shelf) wearable devices, which are adapted to monitor the parameters in order to generate a sustainable wave form, such as a PPG (photoplethysmogram) wave. The present invention is directed to a device, method and system, which utilizes an algorithm that may be implemented in a mobile application using handheld devices, for example, a cloud server or embedded in the wearable device itself. The handheld devices App, computer application or embedded algorithm extracts clinical significant data and communicates with a Cloud server, for example, to help with the treatment. Tools for monitoring and disease management are used to optimize treatment and minimize cost of ER visits, hospitalization and readmission.

The specific characteristics of the PPG wave may be filtered and detected. Changes of hemodynamic parameters affect the PPG wave and the characteristics. These minute changes are detectable and the changes of the hemodynamics are calculated.

The changes of the numerous parameters, such as blood pressure, Cardiac Output and hydration and other parameters may be derived from these calculated parameters.

A feature of the invention is the ability to correctly filter the PPG waveform, detect the PPG's characteristics and calculate the changes of the parameters. The invention is applicable to off-the-shelf wearable devices such as pulse oximeters, wearable bracelets, ring oximeters, for example. The method of the invention may be embedded in any of these devices or any known in the art devices such as an electronic chip sensor, for example.

Cardiac Index divides the CO by the estimated Body Surface Area (BSA) and thereby normalizes the CO value to various body sizes so therefore it is a better estimator of how much blood is supplied to the tissues. Another important aspect is the measurement of CO/CI during periods of rest as well as when the body is undergoing strenuous effort. Many times, during resting, the CO/CI values might seem to be sufficient, but the real test is during effort, when the blood supply is in higher demand. One of the biggest advantages of the wearable device (such as a watch) of the present invention is that it can follow the patient during her/his daily life activities.

The two basic types of heart failure (HF) are diastolic and systolic. Diastolic heart failure happens when the heart cannot properly fill with blood. Systolic heart failure, the more common of the two, occurs when the heart does not efficiently pump blood from the ventricles to the body.

The result of either type of heart failure is a decrease in CO levels, since less blood is pumped from the heart to the body. Decreased CO may also lead to decreased blood pressure.

Many things can lead to heart failure. Systolic HF is commonly caused by a heart attack and/or persistent high blood pressure. Diastolic HF may be a result of systolic HF, dysfunctional heart valves, or a diseased heart lining. Hypertension is one of the most common causes. Other major risk factors are diabetes mellitus, high cholesterol, obesity, and smoking. The continuous measurement of hemodynamics by the system of the present invention will assist the doctor in managing the treatment of the HF patient, keeping her/him away from hospital.

The most important reason why the continuous monitoring of Cardiac Output and other vital signs is relevant for heart failure is that personalizing the management of heart failure and monitoring closely the disease may help to prevent the need for hospitalization whilst optimizing treatment and patient comfort.

There are several guidelines for the diagnosis and management of heart failure. Frequent or continuous monitoring of cardiac output and other vital signs may help to prevent or postpone readmission better adherence and better dosing of medication and will help to provide rapid feedback on lifestyle measures like exercise, diet and supplementation of minerals and vitamins.

There is thus presented a method for monitoring and management of a person's health by measuring changes in hemodynamic parameters. The method includes the steps of inputting personal data into a device worn by a user; calibrating the personal data to set a base line for analysis; monitoring changes in health-related data of the user, recorded by the device and transmitting the health-related data for analysis; analyzing the health-related data for changes in hemodynamic parameters; and outputting the values based on the analysis, thereby allowing a diagnosis of the user's health to be made from the output values.

Furthermore, in accordance with an embodiment of the invention, the step of analysis includes the steps of identifying the features of a PPG (photo plethysmograph) wave produced by the health-related data; determining the heart rate of the user; and processing the quality of the signal produced from the health-related data.

Furthermore, in accordance with an embodiment of the invention, if the signal meets the criteria for quality, the method further includes the steps of producing a matrix having based on the wave feature; performing a linear regression between external blood pressure data source and the calculated values; and subjecting the results to a low pass frequency filter.

Furthermore, in accordance with an embodiment of the invention, the step of monitoring comprises the step of taking samples every “x” seconds, where “x’ is a range of 5-10 seconds. The minimum time period, may be within a range of 30-60 minutes.

Furthermore, in accordance with an embodiment of the invention, the device includes one of a group of devices including a watch, a thumb sensor, a ring oximeter and a thumb oximeter.

Furthermore, in accordance with an embodiment of the invention, the personal data includes at least one of a group of data including gender, age, height, weight, blood pressure and heartrate at rest.

Furthermore, in accordance with an embodiment of the invention, the health-related data includes at least one of group including cardiac input, cardiac output, blood pressure and SPO₂.

Additionally, there is provided, a device and a system for monitoring and management a person's health. The device includes a means for receiving personal data, a means for calibrating the personal data to set a base line for analysis; a means of monitoring changes in health-related data; an analyzer for analyzing the health-related data for changes in hemodynamic parameters and a communications device for transmitting the health-related data to the analyzer.

The system includes a device which includes a means for receiving personal data, a means for calibrating the personal data to set a base line for analysis; and a means of measuring health related data; and an analyzer for analyzing changes in hemodynamic parameters of the health-related data and a communications device for transmitting said health related data to said analyzer.

In the system, the analyzer may be located on a cloud system. The cloud system may store the personal data, the health-related data and changes in the hemodynamic parameters for long-term monitoring and management of thresholds, charts and patient data.

### BRIEF DESCRIPTION OF THE DRAWINGS

The present invention will be understood and appreciated more fully from the following description taken in conjunction with the appended drawings in which:

FIG. 1 is an illustration of exemplary wearable devices in communication with a handheld devices, constructed and operative in accordance with an embodiment of the present invention;

FIG. 2 is a schematic illustration of the wearable device of FIG. 1 in communication with a health management system;

FIG. 3 is a flow chart illustration of the method of monitoring and managing changes in hemodynamic parameters, constructed and operative in accordance with an embodiment of the present invention;

FIGS. 4A and 4B illustrate a typical photo plethysmograph (PPG) wave obtained from a pulse oximeter, used to manage changes in hemodynamic parameters obtained in FIG. 3;

FIGS. 5a and 5b illustrate the PPG (photoplethysmogram) wave of FIG. 4, in time and frequency domains, respectively;

FIG. 6 is a schematic illustration of a typical display of parameters output on a wearable device of FIG. 1;

FIG. 7 is a schematic illustration of alternative wearable devices in communication with a handheld devices embodying the method of FIG. 3;

FIG. 8 is a schematic illustration of alternative wearable devices in which the method of FIG. 3 is embedded; and

FIG. 9 is a schematic illustration of alternative wearable devices in communication with a server embodying the method of FIG. 3.

### DESCRIPTION OF THE INVENTION

The present invention relates to the monitoring and management of heart failure in general and to the monitoring of Cardiac Output (CO) and blood pressure to manage a person's health and reduce the likelihood of heart failure.

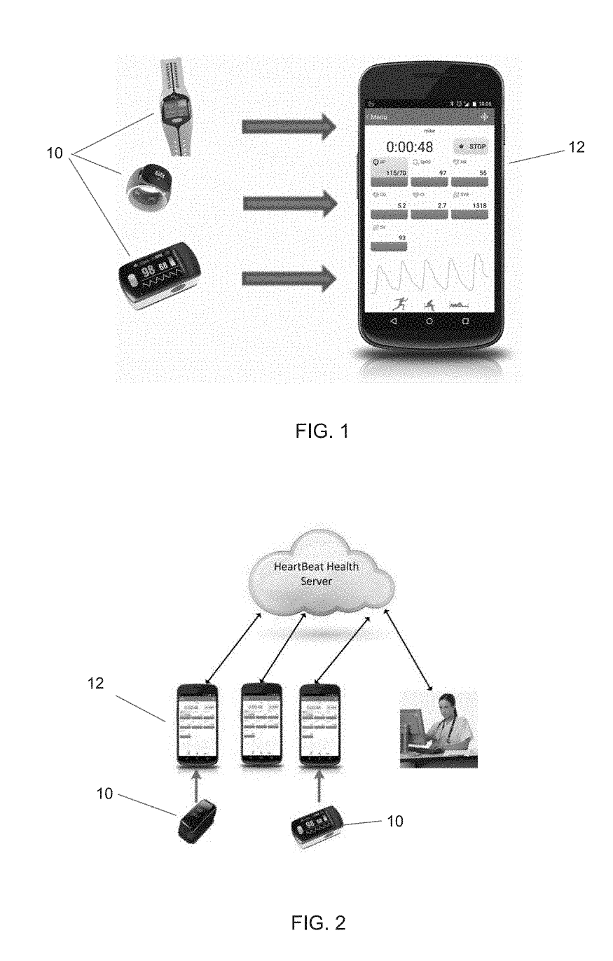

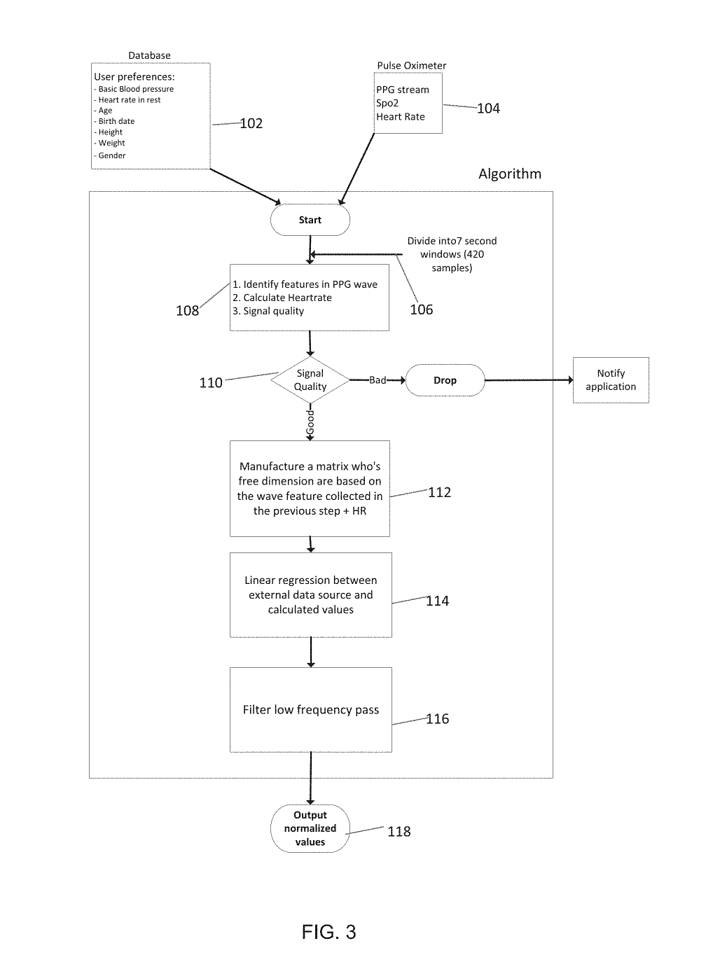

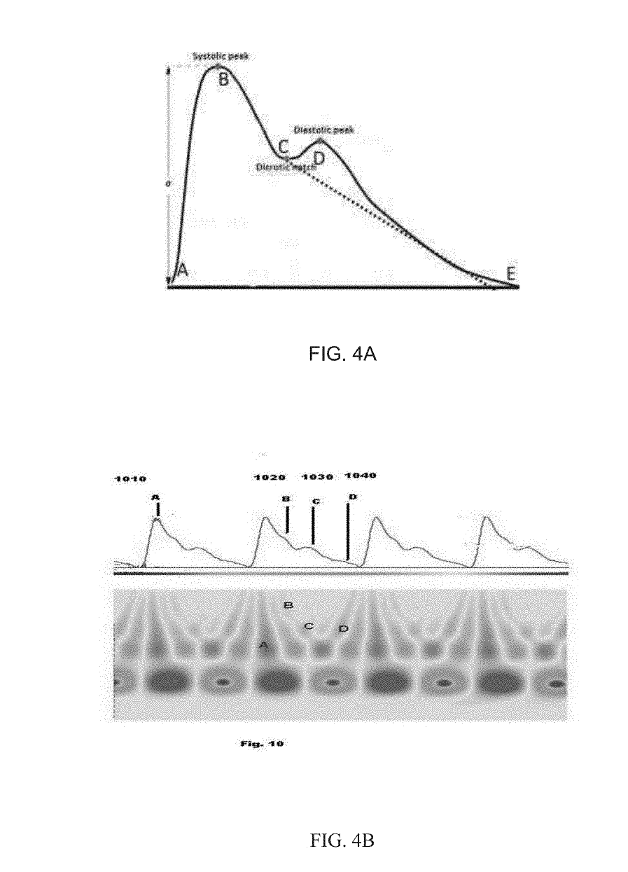

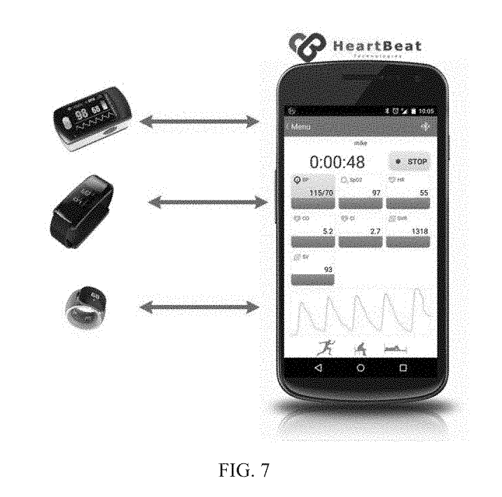

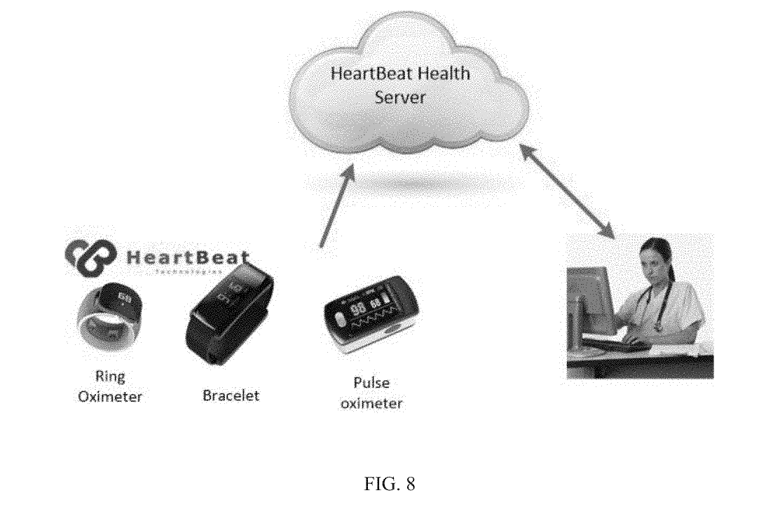

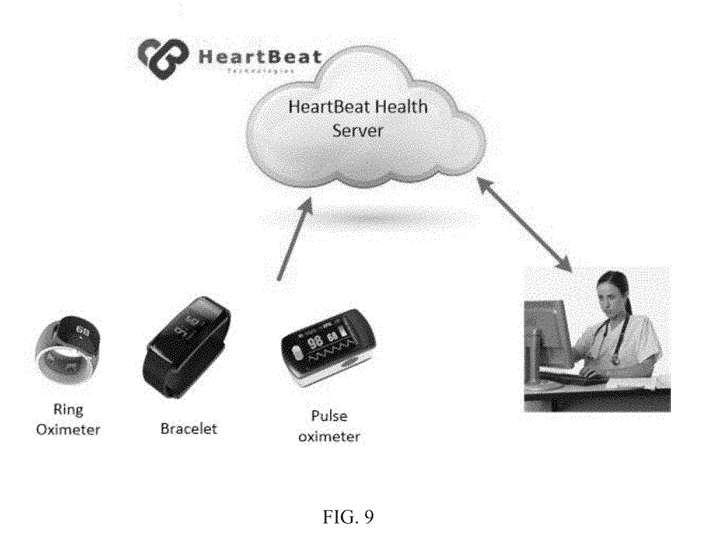

Reference is now made to FIGS. 1-9. FIG. 1 is an illustration of an exemplary wearable devices, generally designated 10 in communication with a handheld devices 12, constructed and operative in accordance with an embodiment of the present invention. FIG. 2 illustrates the wearable device 10 in communication with a health management system. FIG. 3 is a flow chart illustration of the method of monitoring and managing changes in hemodynamic parameters, constructed and operative in accordance with an embodiment of the present invention. FIG. 4 is a schematic illustration of a typical display of parameters output on a wearable device.

The wearable device 10 may comprise any of a group of devices including a watch 10a, having a thumb sensor (not shown) attached thereto, a ring oximeter 10b and a thumb oximeter device. A pulse oximeter device, known in the art monitors the cardiac output and blood pressure of the wearer. In an alternative embodiment, the monitoring sensor may be a reflective sensor integrated on the underside the watch.

The continuous monitoring of CO and continuous blood pressure is based on good quality signal and continuous PPG (photo plethysmograph) wave signals obtained from an optical or pressure based, pulse oximeter or wearable device.

The raw data measured by the pulse oximeter incorporated within any of the wearable devices 10, may be streamed to an application (app) within a mobile handheld devices 12, for example (see FIG. 1). The app runs the algorithm and controls the graphical display. The pulse oximeter may use Bluetooth technology or WIFI, for example, to stream the information to the app.

Reference is now made to the flow chart of FIG. 3.

Since the application monitors the changes of parameters (it does not measure the parameters themselves), a baseline is initially established for each patient. The baseline is established by manually entering general information: gender, age, height, weight, blood pressure and heartrate at rest (step 102). Once established, the application will display the current value of the parameters.

Each session preformed on a patient may be automatically uploaded to a management cloud system for long-term monitoring, thresholds, charts and patient management, as shown in FIG. 2.

The device such as a pulse oximeter supplies health related data (step 104) from the user of the device, the data comprising the PPG (photoplethysmogram) stream, SPO₂ and heart rate, for example. The wave may be analyzed in the following way (steps 106 and 108):
Firstly, the wave is analyzed every few (X) seconds (where X is pre-determined and configured according to the pulse oximeter hardware that supplies the PPG wave—this is referred to as “the PPG wave window” (step 106).
Then, for each PPG wave window, a number (Y) of waves are analyzed (where Y is pre-determined) and configured according to the pulse oximeter hardware that supplies the PPG wave.
The distinct points are identified, the heartrate is calculated and the signal quality noted (step 108),

In the non-limiting example, shown in FIG. 3, the window may be 7 seconds long, allowing for 420 samples in 60 minutes.

A classic PPG wave (shown in FIG. 4A) contains five distinct points (108/1), as follows:
1. Minimum/starting point (marked A and E in FIG. 4A). This is the starting and end point of the wave.
2. Systolic peak (B)
3. Dicrotic notch (C)
4. Dicrotic wave (AKA Diastolic peak) (D)

These points, designated as A, B, C, D and E each have a physiological meaning and are very instructive in calculating heart rate, changes of Cardiac Output, as well as the hydration and continuous blood pressure.

Peak B belongs to the forward moving wave, generated by the left ventricle ejection and the area under this component is proportional to the Cardiac—Output measured in liters/minute. The smaller wave, whose peak is point D, belongs to the returning wave from the iliac and real arteries as well as from the end of the conduit arteries. The three components are proportional to the systemic or total vascular resistance. These reflected waves component depends on the resistance and diminish when the blood vessels dilate as a response to higher flow. (See FIG. 4B)

Once a point is identified, for each wave, the following parameters may be calculated:
Value of A
Value of B
Value of C
Value of D
Value of E
Systol Area=The area under ABCE
Diastol Area=The area under CDE
The Ratio between BC and DC

The heart rate may be calculated (108/2), as follows:

T(E)−T(A)=Beat Period;

where TA is the start of the wave (point A) and TE is the end of the wave (point E)

Heartrate=1/Beat Period×60 (per minute)

After Y waves have been identified and characterized, the signal quality is determined (108/3). During the signal quality process, the following parameters are checked between each wave:
Difference between B values
HR too high or low
Systol Area too small/big
Difference between D values

The sensitivity of each test is determined according to the hardware being used to generate the PPG wave.

If waves are dropped according to the above criteria, that is does not identify all four points (query box 110), the signal wave on the application turns from one color to another (say, green to yellow) to indicate the poor quality of the signal.

Once a good quality sample has been obtained, based on the wave features collected in the previous step, a matrix of data is stored (110).

Then, the waves and detected points are transferred to tables with values and integers and the representative waveforms are subjected to wavelets analysis (step 112).

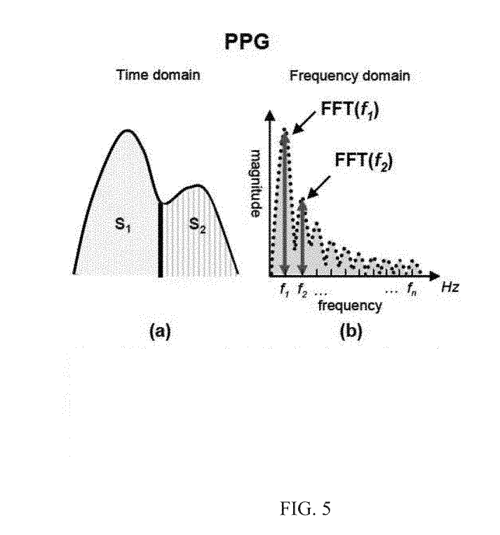

As can be seen in FIGS. 5a and 5b, there are four components that can be identified by decomposing the BP waveforms to its basic ingredients.

The following two values are calculated:

1. Changes of Cardiac output (CO)

CO==(V(AE)−V(CDE))×HR

2. Changes of the MAP (mean arterial pressure)

MAP=V(AE)/V(CDE)

The other parameters are obtained by using the following relations:

MAP=(SYS+2DIA)/3

In the next stage of the process, a linear regression between external blood pressure data source and the calculated values is performed (step 114).

In the next stage, the results are subjected to a low pass frequency filter. In this step spikes from the calculated parameters caused by external interference such as sudden movement of the sensor are removed (step 116).

In addition, the following values may be calculated:

SVR=80*MAP/CO

SV=CO/HR

CI (Cardiac Index)=CO/BSA(Body surface area=([Height(cm)×Weight(kg)]/3600)½)

Steps 106-116 are performed every ‘N” seconds (N being a pre-determined time) and a new set of parameters are generated.

The signal processing takes place within the handheld devices in real time, which minimizes the amount of communication with the “cloud” and allows the handheld devices to work independently, even in places and situations where communication to the “cloud” is unavailable.

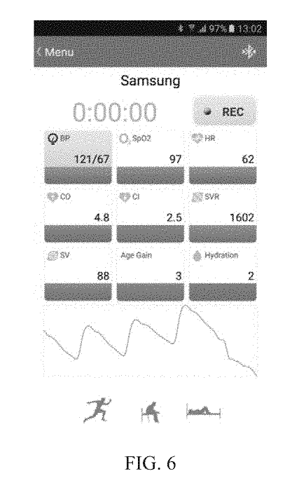

FIG. 6 illustrates a typical output display, such as may appear on a handheld devices. The presentation of the processed data is explained in more detail below. The main objective of the display of the invention is to present all relevant data in a graphic concise way, so that the doctor or other medical person may obtain a picture of the patient's status, trend over time and relationship among all components, in order to save the doctor's time and promote treatment that is more efficient.

There are many different ways to categorize heart failure (HF), including the following non-limiting examples:
1. The side of the heart involved (left HF versus right HF). Right HF compromises pulmonary flow to the lungs. Left HF compromises aortic flow to the body and brain. Mixed presentations are common; left HF often leads to right HF in the longer term.
2. Whether the abnormality is due to insufficient contraction (systolic dysfunction), or due to insufficient relaxation of the heart (diastolic dysfunction), or to both. Whether the problem is primarily increased venous back pressure (preload), or failure to supply adequate arterial perfusion (afterload),
3. Whether h abnormality is due to low CO with high systemic vascular resistance or high CO with low vascular resistance (low-output HF vs. high-output HF).
4. The degree of functional impairment conferred by the abnormality (as reflected in the New York Heart Association Functional Classification
5. The degree of coexisting illness: that is HF/systemic hypertension, HF/pulmonary hypertension, HF/diabetes, HF/renal failure, for example.

The doctor may make his diagnosis based on his interpretation of the measurements. Once the results are sent to the cloud server, the treating doctor may enter her/his diagnosis and treatment into the patient's file.

This information may be used, subject to privacy considerations, such as not mentioning the patients name and other sensitive data, in order to generate statistical data related to heart failure in the general population.

A cloud server and inference engine, which combines data, diagnosis and treatment of many patients to figure out the relative importance and contribution of each component to the total health score of the patient, leading to accumulated knowledge for optimizing diagnostics and treatment may be developed as a by-product from the present invention. These statistical inferences will be used for recommendation and information purposes only, and will not affect the diagnosis or treatment decisions.

The data-display could play a major role in the usefulness of the personal server to the monitoring and disease management providers. As mentioned above, the representation of the data and its graphical presentation, as well as specific calculations save the doctor's time and do not involve any medical interpretation or decisions. The diagnosis and treatment decisions are the doctor's responsibility

Prior to activating the Graphical User Interface (GUI), an input table should contain the following data:
1. ID of patient—integer number
2. Height (in meters)—e.g. 1.73
3. Weight (in Kg)—e.g. 66 Kg
4. BSA (Body Surface Area)—computed from 1 and 2, may be computed from the Mosteller Formula, below

### The Mosteller Formula

BSA(m²)=([Height(cm)×Weight(kg)]/3600)^{1/2}
e.g. BSA=SQRT((cm*kg)/3600) or in inches and pounds:

BSA(m²)=([Height(in)×Weight(lbs)]/3131)^{1/2}
5. List of parameters to be displayed (as determined by the doctor) e.g. CO (Cardiac Output), CI=CO/BSA, SpO2, BP. Activity (calories or kW—computed from 3D accelerometer), Respiration (Res per minute), temperature, ECG, for example.
6. Series of numbers expressing relative importance for each chosen parameter (for example: CO—0.3; BP—0.15, . . . )
7. For each chosen parameter—the range of normal values, for example CO—4-6 L/min: BP—120-100 Sys; 60-80 Diastolic'
8. Alarm thresholds for each chosen parameter

This table need be filled in only once, and may be populated either by the treating physician or from known norms. Some values can be sent from devices like Bluetooth Weight Scale, or computed from other entries, such as, BSA)

This table may be changed by the physician at any time, depending on the progress of the disease.

The exemplary embodiment of FIG. 64 illustrates how the display may appear on the handheld devices.

### Top Pane:

Devices—will allow the user to add devices, such as SpO2, BP, ECG, Weight Scale (body composition), Temp, and Respiration, for example.

### Main Pane Buttons

Shows you the measured parameters in real time: HR (heart rate), SpO2, Blood Pressure, Cardiac Output, Cardiac Index, SVR (Systemic Vascular Resistance) and SV (Stroke Volume)

The colors of the buttons change as the parameters exceed or drop beneath pre-defined thresholds.

### PPG Graph Pane:

Displays the PPG graph obtained from the Pulse Oximeter.

It should be emphasized that the GUI only reflects the patient data, either recorded or computed, and the physician's preferences (relative importance) but does not make any clinical judgment.

It will be appreciated that the present invention is not limited by what has been described hereinabove and that numerous modifications, all of which fall within the scope of the present invention, exist. Rather the scope of the invention is defined by the claims, which follow:

1. A method for monitoring and management of a person's health, comprising the steps of:
inputting personal data into a device worn by a user;
calibrating said personal data to set a base line for analysis;
monitoring changes in health-related data of said user, recorded by said device and transmitting the health-related data for analysis;
analyzing said health related data for changes in hemodynamic parameters; and
outputting the values based on said analysis, thereby allowing a diagnosis of the user's health to be made from the output values.

2. The method according to claim 1, wherein said step of analysis comprises the steps of:
identifying the features of a PPG (photo plethysmograph) wave produced by the health related data;
determining the heart rate of the user; and
processing the quality of the signal produced from said health related data.

3. The method according to claim 2, wherein if the signal meets the criteria for quality, further comprising the steps of:
producing a matrix having based on the wave feature;
performing a linear regression between external blood pressure data source and the calculated values; and
subjecting the results to a low pass frequency filter.

4. The method according to claim 1, wherein said step of monitoring comprises the step of:
taking samples every ‘N’ seconds, where ‘N’ is a range of 5-10 seconds.

5. The method according to claim 4, wherein said step of monitoring comprises the step of:
for a minimum time period, said time periods being within a range of 30-60 minutes.

6. The method according to claim 1, wherein said device comprises one of a group of devices including a watch, a thumb sensor, a ring oximeter and a thumb oximeter

7. The method according to claim 1, wherein said personnel data comprises at least one of a group of data including gender, age, height, weight, blood pressure and heartrate at rest.

8. The method according to claim 1, wherein said health related data comprises at least one of group including cardiac input, cardiac output, blood pressure and SPO₂.

9. The device for monitoring and management a person's health, comprising:
a means for receiving personal data, wherein said personal data comprises at least one of a group of data including gender, age, height, weight, blood pressure and heartrate at rest;
a means for calibrating said personal data to set a base line for analysis;
a means of monitoring changes in health related data;
an analyzer for analysing said health related data for changes in hemodynamic parameters; and
a communications device for transmitting said health related data to said analyzer.

10. The device according to claim 9, wherein said device comprises one of a group of devices including a watch, a thumb sensor, a ring oximeter and a thumb oximeter.

11. The device according to claim 9, wherein said means of measuring health related data comprises at least one of a group including a cardiac output monitor, a blood pressure measurer; and a SPO₂ measurer.

12. The device according to claim 9, wherein said analyzer s configured to:
identify the features of a PPG (photo plethysmograph) wave produced by the health related data;
determine the heart rate of the user; and
process the quality of the signal produced from said health related data.

13. The device according to claim 12, wherein said analyzer is further configured to:
produce a matrix having based on the wave feature;
perform a linear regression between external blood pressure data source and values analyzed; and
subject the results to a low pass frequency filter.

14. A system for monitoring and management of a person's health, comprising:
a device comprising:
a means for receiving personal data, wherein said personal data comprises at least one of a group of data including gender, age, height, weight, blood pressure and heartrate at rest;
a means for calibrating said personal data to set a base line for analysis; and
a means of measuring health related data;
an analyzer for analysing changes in hemodynamic parameters of said health related data; and
a communications device for transmitting said health related data to said analyzer.

15. The system according to claim 14, wherein said analyzer is located on a cloud system and wherein said cloud system stores said personal data, said health related data and changes in the hemodynamic parameters for long-term monitoring and management of thresholds, charts and patient data.

16. The system according to claim 14, wherein said device comprises one of a group of devices including a watch, a thumb sensor, a ring oximeter and a thumb oximeter.

17. The system according to claim 14, wherein said means of measuring health related data comprises at least one of a group including a cardiac output monitor, a blood pressure measurer; and a SPO₂ measurer.

18. The system according to claim 14, wherein said analyzer is configured to:
identify the features of a PPG (photo plethysmograph) wave produced by the health related data;
determine the heart rate of the user; and
process the quality of the signal produced from said health related data.

19. The system according to claim 18, wherein said analyzer is further configured to:
produce a matrix having based on the wave feature;
perform a linear regression between external blood pressure data source and values analyzed; and
subject the results to a low pass frequency filter.
